(19) United States
(12) Patent Application Publication
(10) Pub. No.: US 2026/0034255 A1
(43) Pub. Date: Feb. 5, 2026

(54) Disinfection Device and Associated Disinfection Process

(71) Applicant: GERMITEC, Bordeaux (FR)

(72) Inventors: Fanny POULON, Bures Sur Yvette (FR); Jérémy DALLARD, Talence (FR); Maxime BERTHIAS, Cadarsac (FR); Aimrane HAMDOU, Bordeaux (FR)

(21) Appl. No.: 19/283,704

(22) Filed: Jul. 29, 2025

(30) Foreign Application Priority Data
Jul. 31, 2024 (FR) FR2408460

(51) Int. Cl. A61L 2/10; A61L 2/26

(52) U.S. Cl. CPC A61L 2/10; A61L 2/26; A61L 2202/11; A61L 2202/122; A61L 2202/24

(57) ABSTRACT

The invention relates to a disinfection device (10) for a medical instrument comprising a disinfection volume (24) delimited by at least one wall (22A-E), a UV-C radiation generation device (26) configured to generate UV-C radiation in the disinfection volume (24), and a support (28) configured to hold the entire medical instrument inside the disinfection volume (24), the support (28) being transparent to UV-C radiation.

The present invention relates to a disinfection device for a medical instrument and an associated disinfection method.

To avoid any contamination, it is necessary to disinfect a medical instrument between uses.

For this purpose, it is known from the prior art to disinfect medical instruments in disinfection devices defining a closed disinfection volume. Such devices comprise, for example, a device for generating UV (ultraviolet) radiation allowing, in particular, high-level disinfection of these medical instruments.

Medical instruments, such as medical probes, typically comprise at least one active part and, preferably, at least one connecting part in the form of a cable.

It is known from the prior art to suspend these medical instruments in the disinfection device, for example, using a suspension arm comprising, for example, a knob or a ring adapted to hold a portion of the connecting part.

Generally, in such disinfection devices, at least said portion of the connecting part is not disinfected since it is outside the disinfection volume.

Such disinfection is therefore not entirely satisfactory, as there is a risk of contamination in this non-disinfected area.

The aim of the invention is to propose a disinfection device allowing improved disinfection of the medical instrument.

To this end, the invention relates to a disinfection device for a medical instrument comprising:
a disinfection volume delimited by at least one wall,
a UV-C radiation generation device configured to generate UV-C radiation in the disinfection volume, and
a support configured to hold the entire medical instrument inside the disinfection volume, the support being transparent to UV-C radiation.

By means of the support, the medical instrument is held entirely within the disinfection volume, preventing any part of the instrument from not being disinfected.

Furthermore, the feature that the support is transparent to UV-C radiation ensures that even the portions of the medical instrument that are directly held by the support, particularly those in contact with it, are reached by the UV-C radiation, and thus disinfected.

According to other advantageous aspects of the invention, the disinfection device comprises one or more of the following features, taken individually or in any technically possible combination:
The support has a transmittance to UV-C radiation greater than 30%, and preferably greater than 50%.
The support comprises at least one pair of elements comprising a first element and a second element, the medical instrument being intended to be held by the first and second elements.
The at least one wall comprises at least a first zone and a second zone different from the first zone, the first element being fixed to the first zone and the second element being fixed to the second zone.
The support comprises two pairs of elements.
The first and/or second element of the pair or of each pair of elements comprises a plate comprising a lateral insertion opening to allow the insertion of a portion of the medical instrument.
The at least one wall comprises a bottom, the support comprising a third element fixed to the bottom, the medical instrument being intended to be held by the third element.
The support is made of quartz, preferably synthetic quartz.

The invention also relates to a method for disinfecting a medical instrument comprising the following steps:
a. providing a device as described above, and
b. positioning the entire medical instrument inside the disinfection volume, the medical instrument being held by the support.

Advantageously, the medical instrument comprises a handle and preferably at least one connector, the handle of the medical instrument being held by one of the first elements, advantageously the connector of the medical instrument being held by one of the second elements.

The invention will become clearer upon reading the following description, given solely by way of non-limiting example, and made with reference to the drawings wherein:

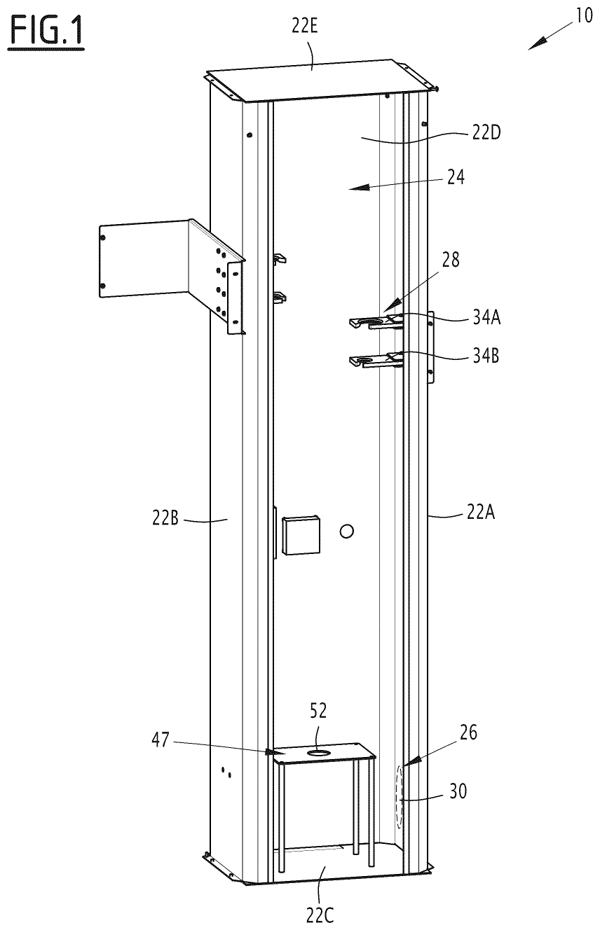

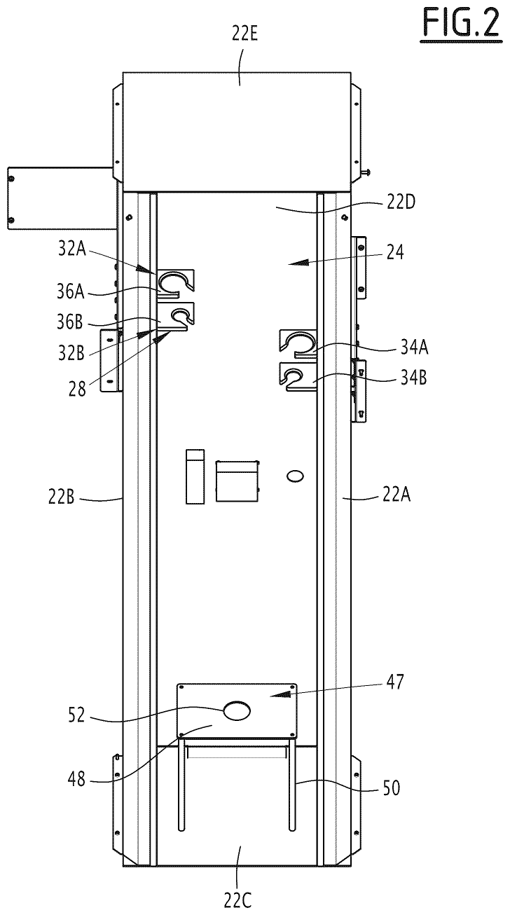

FIGS. 1 and 2 are schematic perspective views of an example of a disinfection device according to the invention;

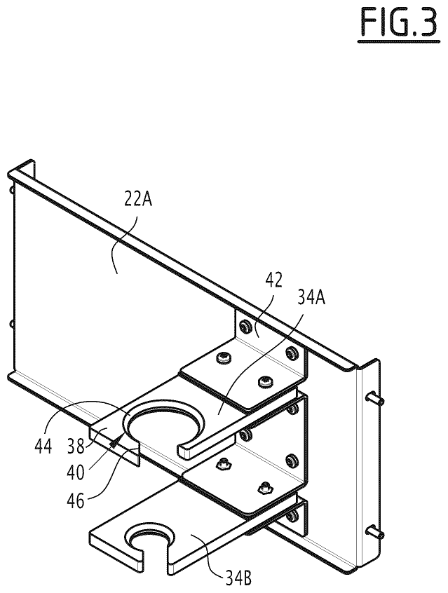

FIG. 3 is a schematic perspective view of an example of two elements of the support according to the invention; and

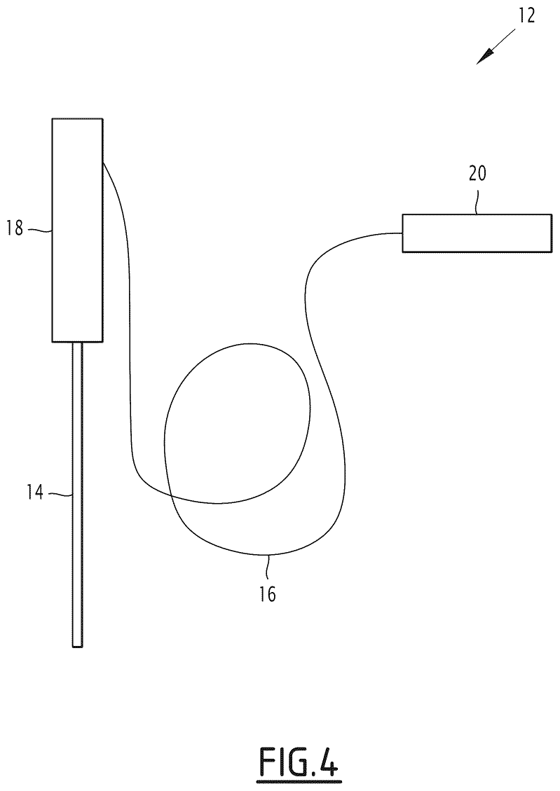

FIG. 4 is a schematic view of an example of a medical instrument suitable for being disinfected by the disinfection device shown in FIG. 1.

FIGS. 1 and 2 illustrate an example of a disinfection device 10 according to the invention. The disinfection device 10 is configured to disinfect a medical instrument 12 in its entirety.

Disinfection of the medical instrument 12 in its entirety should be understood to mean disinfection of all parts of the medical instrument 12 in the disinfection device 10.

FIG. 4 illustrates an example of a medical instrument 12 suitable for being disinfected by the disinfection device 10.

The medical instrument 12 comprises, for example, an active part 14. The active part 14 is, for example, a tube intended to be inserted into the patient, for example into a nostril of the patient.

Preferably, the medical instrument 12 comprises a handle 18 connected to the active part 14. The handle 18 is advantageously fixed to one end of the active part 14 and is intended to be grasped by a user to facilitate the insertion of the active part 14 into the patient.

The medical instrument 12 further preferably comprises a connecting part 16 and even more preferably also a connector 20.

The connecting part 16 is, for example, a connecting cable connecting the active part 14 to the connector 20.

The connector 20 is, for example, configured to be connected to a measurement and/or display unit, in particular to collect and/or display information captured by the active part 14.

The medical instrument 12 is, for example, a medical probe, in particular an ultrasound medical probe, and more particularly a transesophageal ultrasound (TEE) medical probe or an endoscopic medical probe, for example of the “otorhinolaryngology” (ENT) type. The medical probe is, for example, a pediatric or adult probe.

The disinfection device 10 comprises a disinfection volume 24 delimited by at least one wall, a UV-C radiation generation device 26, and a support 28.

Advantageously, the at least one wall comprises a bottom 22C.

Preferably, the at least one wall comprises at least a first zone and a second zone different from the first zone.

In a preferred embodiment, the disinfection device 10 comprises a plurality of walls 22A, 22B, 22C, 22D, 22E defining between them the disinfection volume 24. More particularly, the walls 22A, 22B, 22C, 22D, 22E, 22F define between them a closed disinfection volume 24, for example, parallelepiped in shape.

In the embodiment of the invention illustrated in FIGS. 1 and 2, the plurality of walls comprises, for example, a first lateral wall 22A and a second lateral wall 22B opposite each other, a bottom 22C, and a cover 22E opposite the bottom 22C, and also a base 22D defining a third lateral wall and a door (not shown) facing the base 22D.

For example, the first lateral wall 22A defines the first zone and the second lateral wall 22B defines the second zone.

In particular, the cover 22E and the bottom 22C are, for example, perpendicular to the two lateral walls 22A, 22B. Similarly, the base 22D and the door are, for example, perpendicular to the two lateral walls 22A, 22B, the cover 22E, and the bottom 22C.

In an unillustrated variant, the disinfection device 10 comprises a single wall 22 delimiting the disinfection volume 24, which is, for example, cylindrical or spherical.

The UV-C radiation generation device 26 is configured to generate UV-C radiation in the disinfection volume 24.

For example, the UV-C radiation generation device 26 comprises a plurality of UV-C radiation sources 30 arranged inside the disinfection device 10, and, for example, on at least one of the walls 22, for example, at least one of the lateral edges of the base 22D as illustrated in FIG. 1.

Each UV-C radiation source 30 is, for example, a mercury lamp generating UV-C radiation. In particular, each mercury lamp 30 is capable of using mercury to excite a gas and produce UV-C ultraviolet radiation. UV-C radiation should be understood to mean radiation characterized by a wavelength between 200 and 280 nm.

Alternatively, each UV-C radiation source 30 is a LED (Light-Emitting Diode) lamp.

The support 28 is configured to hold the entire medical instrument 12 inside the disinfection volume 24.

In other words, when the medical instrument 12 is held by the support 28, all the elements constituting the medical instrument 12 are arranged inside the disinfection volume.

In particular, the active part 14, the connecting part 16, the handle 18, and the connector 20 are entirely held inside the disinfection volume 24.

More particularly, the two ends of the connecting part 16 are inside the disinfection volume 24 when the medical instrument 12 is held by the support 28.

The support 28 is transparent to UV-C radiation.

In particular, the support 28 has a transmittance to UV-C radiation greater than 30%, and preferably greater than 50%, and even more preferably greater than 90%.

“Transmittance” should be understood to mean the ratio of radiation transmitted by the support 28 to the incident radiation on the support 28.

In other words, at least 30% of the UV-C radiation incident on the support 28 is transmitted through the support 28.

The support 28 is preferably made of quartz, preferably synthetic quartz, such as, for example, a JGS1-category optical quartz glass.

Alternatively, the support 28 is made of a polymer, preferably a cyclic olefin copolymer (COC), in particular TOPAS® 8007X10, or a polymethyl pentene, such as TPX™ RT18, or fluorinated ethylene-propylene.

As illustrated in FIGS. 1 and 2, the support 28 comprises at least one pair of elements 32A, 32B, and preferably two pairs of elements 32A, 32B.

Each pair of elements 32A, 32B comprises a first element 34A, 34B, and a second element 36A, 36B.

Each first element 34A, 34B, and second element 36A, 36B is configured to hold a portion of the medical instrument 12, for example, the handle 18 or the connector 20.

The first element 34A, 34B, and the second element 36A, 36B of the same pair 32A, 32B are preferably identical.

Each first element 34A, 34B is, for example, fixed to the first lateral wall 22A.

Each second element 36A, 36B is, for example, fixed to the second lateral wall 22B, 20 preferably at a height relative to the bottom 22C different from the first element 34A, 34B of the same pair.

As illustrated in FIG. 3, each first element 34A, 34B, and/or each second element 36A, 36B comprises a plate 38 comprising a lateral insertion opening 40 to allow the insertion of a portion of the medical instrument 12.

In particular, the plate 38 is, for example, a plate made of a material transparent to UV-C radiation, for example, quartz.

In the example illustrated in FIG. 3, the plate 38 is substantially rectangular in shape.

For example, the plate 38 has a thickness between 0.5 cm and 2 cm.

As visible in FIG. 3, the plate 38 is, for example, fixed to the corresponding lateral wall 22A, 22B by fixing brackets 42 fixed, for example, by screws.

The lateral insertion opening 40 is preferably arranged on the plate 38 to allow the insertion of a portion of the medical instrument 12 and its retention.

The lateral insertion opening 40 comprises, for example, a circular bore 44 and a recess 46.

The circular bore 44 preferably has a diameter suitable for holding a portion of the medical instrument 12, such as the handle 18 and/or the connector 20. The circular bore 44 has, for example, a diameter between 20 mm and 50 mm.

As visible in FIG. 3, the recess 46 is preferably arranged to connect an edge of the plate 38 to the circular bore 44, to allow the insertion of the portion of the medical instrument 12 into the circular bore 44.

The recess 46 is advantageously oriented at an angle of about 45° relative to the edges of the plate 38.

Advantageously, the lateral insertion opening 40 of the first element 34A, 34B, and the second element 36A, 36B of the same pair of elements 32A, 32B are identical.

Preferably, in the embodiment wherein the support 28 comprises two pairs of elements 32A, 32B, the lateral insertion openings 40 of the first elements 34A, 34B, respectively of the second elements 36A, 36B, of the two pairs are different, and in particular have different dimensions. Such a difference allows greater adaptability and, in particular, the retention of medical instruments 12 with varied dimensions and configurations.

Furthermore, the support 28 preferably comprises a third element 47 fixed to the bottom 22C.

The third element 47 is preferably suitable for holding a portion of the medical instrument 12, and in particular the active part 14 of the medical instrument 12, at a distance from the walls 22 of the disinfection device 10.

The third element 47 comprises, for example, a plate 48 fixed to the bottom 22C via fixing feet 50.

More particularly, the plate 48 is substantially parallel to the bottom 22C and is held at a distance from it by the fixing feet 50.

The plate 48 is, for example, a plate made of a material transparent to UV-C radiation, for example, quartz.

Advantageously, the plate 48 comprises a circular bore 52 advantageously of a diameter suitable for receiving the active part 14 of the medical instrument 12. The diameter is, for example, between 30 mm and 50 mm.

A method for disinfecting the medical instrument 12 in the disinfection device 10 will now be described.

Firstly, a disinfection device 10 as described above is provided.

Then, the medical instrument 12 intended to be disinfected is positioned in the disinfection volume 24 of the disinfection device 10.

In particular, the medical instrument 12 is positioned entirely inside the disinfection volume 24, the medical instrument 12 being held by the support 28.

In particular, the operator inserts a portion of the medical instrument 12 into the circular bore 44 of at least one of the first elements 34A, 34B, and at least one of the second elements 36A, 36B.

More precisely, in the preferred embodiment illustrated in the figures, the handle 18 of the medical instrument 12 is, for example, held by one of the first elements 34A, 34B, and the connector 20 of the medical instrument 12 is held by one of the second elements 36A, 36B. The connecting part 16 extends between these two elements.

Depending on the medical instrument 12 to be disinfected, the first element 34A holding the handle 18 and the second element 36A holding the connector 20 belong to the same pair of elements or different pairs.

The active part 14 is preferably held in the third element 47 of the support 28. In particular, the active part 34 is inserted into the circular bore 52 arranged in the plate 48 of the third element 47.

The door (not shown) is then closed.

A disinfection cycle by UV-C radiation generated by the UV-C radiation generation device 26 is then implemented.

Such a disinfection device 10 is particularly advantageous for the disinfection of medical instruments 12, and in particular medical probes.

In particular, by means of the support 28, the medical instrument 12 is held entirely in the disinfection volume 12, preventing any portion of the instrument from not being disinfected.

Furthermore, the feature that the support 28 is transparent to UV-C radiation ensures that even the portions of the medical instrument 12 that are directly held by the support 28, particularly those in contact with it, are reached by the UV-C radiation, and thus disinfected.

Moreover, the embodiment using a support 28 in quartz is particularly advantageous, as this material guarantees a transmittance to UV-C radiation greater than 90%. Furthermore, such a material has very good mechanical robustness, and its UV-C radiation transmission capabilities remain unchanged even after hundreds of hours of exposure.

Furthermore, the support 28 is suitable for holding different types and sizes of medical instruments 12.

The person skilled in the art will understand that the previously described embodiments and variants can be combined with each other provided they are technically compatible.

1. A disinfection device for a medical instrument comprising:
a disinfection volume delimited by at least one wall,
a UV-C radiation generation device configured to generate UV-C radiation in the disinfection volume, and
a support configured to hold the entire medical instrument inside the disinfection volume, the support being transparent to UV-C radiation.

2. The device according to claim 1, wherein the support has a transmittance to UV-C radiation greater than 30%.

3. The device according to claim 2, wherein the support has a transmittance to UV-C radiation greater than 50%.

4. The device according to claim 1, wherein the support comprises at least one pair of elements comprising a first element and a second element, the medical instrument being intended to be held by the first and second elements.

5. The device according to claim 4, wherein the at least one wall comprises at least a first zone and a second zone different from the first zone, the first element being fixed to the first zone and the second element being fixed to the second zone (22B).

6. The device according to claim 4, wherein the support comprises two pairs of elements.

7. The device according to claim 4, wherein the first and/or second element of the pair or of each pair of elements comprises a plate comprising a lateral insertion opening to allow the insertion of a portion of the medical instrument.

8. The device according to claim 1, wherein the at least one wall comprises a bottom, the support comprising a third element fixed to the bottom, the medical instrument being intended to be held by the third element.

9. The device according to claim 1, wherein the support is made of quartz.

10. The device according to claim 9, wherein the support is made of synthetic quartz.

11. A method for disinfecting a medical instrument comprising the following steps:
a. providing a device according to claim 1, and
b. positioning the entire medical instrument inside the disinfection volume, the medical instrument being held by the support.

12. The method according to claim 11, wherein the medical instrument comprises a handle, a disinfection device for a medical instrument comprising a disinfection volume delimited by at least one wall, a UV-C radiation generation device configured to generate UV-C radiation in the disinfection volume, and a support configured to hold the entire medical instrument inside the disinfection volume, the support being transparent to UV-C radiation, wherein the support comprises at least one pair of elements comprising a first element and a second element, the medical instrument being intended to be held by the first and second elements, the handle of the medical instrument being held by one of the first elements.

13. The method according to claim 12, wherein the medical instrument comprises at least one connector, the connector of the medical instrument being held by one of the second elements.
